(19) United States
(12) Patent Application Publication
(10) Pub. No.: US 2025/0120412 A1
(43) Pub. Date: Apr. 17, 2025

(54) MULTI-LAYER FERMENTATION TANK FOR TEA LEAVES AND USE METHOD THEREOF

(71) Applicant: Zhejiang University, Hangzhou City (CN)

(72) Inventors: Xiaoli LI, Hangzhou City\ (CN); Yong HE, Hangzhou City,\ (CN); Qinghai HE, Hangzhou City (CN); Xuelun LUO, Hangzhou City (CN); Yuying ZHANG, Hangzhou City (CN); Huahao YU, Hangzhou City (CN)

(21) Appl. No.: 18/911,692

(22) Filed: Oct. 10, 2024

(30) Foreign Application Priority Data
Oct. 16, 2023 (CN) 202311329653.X

(51) Int. Cl. A23F 3/08; C12M 1/00; C12M 1/34; C12M 3/00

(52) U.S. Cl. CPC A23F 3/08; C12M 23/26; C12M 23/34; C12M 23/48; C12M 23/52; C12M 41/14; C12M 41/34

(57) ABSTRACT

Provided are a multi-layer fermentation tank for tea leaves and a use method thereof. The multi-layer fermentation tank for tea leaves includes a tank body, a placement assembly, a heater, and an oxygen supply machine. The placement assembly includes a support frame, and a tray. The support frame includes a turnover plate, laminate groups located at upper and lower sides of the turnover plate, and a top cover located above the laminate group at the upper side. A telescopic connection mechanism is arranged among the turnover plate, the laminate and the top cover, an upper surface of each of the turnover plate and the laminate is provided with a placement groove, the tray is placed in the placement groove, and a positioning member is arranged between the tray and the placement groove. Gas holes are formed in each of the turnover plate, the laminate, the tray, and the top cover.

### CROSS-REFERENCE TO RELATED APPLICATION

This patent application claims the benefit and priority of Chinese Patent Application No. 202311329653.X, filed with the China National Intellectual Property Administration on Oct. 16, 2023, the disclosure of which is incorporated by reference herein in its entirety as part of the present application.

### TECHNICAL FIELD

The present disclosure relates to the technical field of tea leaves processing, in particular to a multi-layer fermentation tank for tea leaves, and a use method thereof.

### BACKGROUND

The fermentation of tea is an important link in the process of tea processing. In the prior art, fermentation tank is mostly used to ferment tea leaves, and the tea leaves are stacked in the fermentation tank. However, the tea leaves at the lower layer are inadequately fermented due to less contact with oxygen. A multi-layer fermentation device for tea leaves as disclosed in China patent CN219422086U includes a tank body. Both inner walls of a tank body are provided with chutes, sliders are slidably connected in to both chutes, and a placement plate is fixedly connected to each of opposite surfaces of the two sliders. A support column is fixedly connected above the placement plate, and a fermentation box is fixedly connected above the support column. According to the patented technology, multiple fermentation boxes arranged up and down are arranged in the tank body, and a stirring structure for stirring tea leaves is placed in each fermentation box to solve the problem that the tea leaves at the lower layer are inadequately fermented when the tea leaves are stacked. The defects of the above patented technology are as follows: (1) the multi-layered placement plates can divide the tank body into several parts which are independent from each other up and down, making the gas mobility in the whole tank body poor. (2) Each fermentation box needs to be equipped with a stirring structure, leading to complicated overall structure and inconvenient laying of tea leaves in the fermentation box.

### SUMMARY

An objective of the present disclosure is to provide a multi-layer fermentation tank for tea leaves and a use method thereof, which are used to solve the problems of poor gas fluidity in the tank body, inconvenient taking and placing of tea leaves, and inadequate fermentation of tea leaves at a lower layer in the existing multi-layer fermentation device for tea leaves.

The technical solution adopted by the present disclosure for solving the technical problem is as follows. A multi-layer fermentation tank for tea leaves includes a tank body, a placement assembly, a heater, and an oxygen supply machine. A front wall of the tank body is provided with a tank door and the heater and the oxygen supply machine are arranged in the tank body. The heater is used to heat the inside of the tank body, and the oxygen supply machine is used to supply oxygen into the tank body. The placement assembly includes a support frame, and a tray. The support frame includes a turnover plate, laminate groups located at upper and lower sides of the turnover plate, and a top cover located above the laminate group at the upper side. A telescopic connection mechanism is arranged among the turnover plate, the laminate, and the top cover. An upper surface of each of the turnover plate and the laminate is provided with a placement groove, the tray is located in the placement groove, and a positioning member is arranged between the tray and the placement groove. Gas holes are formed in each of the turnover plate, the laminate, the tray, and the top cover. A motor for driving the turnover plate to rotate in a vertical plane is arranged in the tank body.

Further, the tank body is internally provided with a first box body and a second box body arranged left and right. The support frame is located between the first box body and the second box body, and each of left and right side walls of the turnover plate is provided with a support shaft. The support shaft at a left end of the turnover plate is rotatably connected to the first box body, and the support shaft at a right end of the turnover plate is rotatably connected to the second box body. The motor is arranged in the first box body, and has an output end fixedly connected to the support shaft at the left end of the turnover plate.

Further, two telescopic connection mechanisms are arranged left and right, and the support frame is located between the two telescopic connection mechanisms.

Further, the telescopic connection mechanism includes a connecting rod group, and a driving member. Multiple connecting rod groups are arranged in sequence, and each connecting rod group includes two connecting rods which are crossed and hinged. Corresponding end portions of two adjacent connecting rod groups are hinged, an end portion of one connecting rod of the connecting rod group is hinged with the corresponding top cover, laminate, or turnover plate, and an end portion of the other connecting rod in the connecting rod group is in sliding connection with the corresponding top cover, laminate, or turnover plate. The driving member is arranged on the turnover plate to drive the two connecting rods of the connecting rod group to swing relative to each other and maintain the two connecting rods at a fixed included angle.

Further, an end portion of the other connecting rod of the connecting rod group is hinged with a slider. Each of left and right side walls of the top cover, the laminate or the turnover plate is provided with a chute, and the slider is in sliding connection with the corresponding chute.

Further, the driving member is an electric telescopic rod, a main body of the driving member is fixed into a side wall of the turnover plate, and the slider in sliding connection with the turnover plate is fixed to a telescopic rod of the driving member.

Further, the positioning member includes a magnet, and an iron sheet. The magnet is embedded in an inner wall of the placement groove, the iron sheet is embedded in an outer wall of the tray, and the iron sheet is in contact and attracted with a corresponding magnet after the tray is placed into the placement groove.

Further, the bottom of the top cover is provided with a square groove.

Further, an elastic sheet is provided between two connecting rods of the connecting rod groups.

The present disclosure further provides a use method of a multi-layer fermentation tank for tea leaves, including the following steps:
(1) placing tea leaves on a tray, placing the tray on each of a laminate and a turnover plate, and closing a tank door;
(2) turning on a heater and an oxygen supply machine to ferment the tea leaves;
(3) after fermenting for a period of time, driving, by a driving member, a connecting rod group to swing to make a top cover in contact with the laminate, two adjacent laminates in contact with each other, and the laminate in contact with the turnover plate, and turning on a motor to overturn a support frame by 180 degrees; then driving, by the driving member, the top cover to separate from the laminate, the two adjacent laminates to separate from each other, and the laminate to separate from the turnover plate, thus interchanging positions of the tea leaves in the tray up and down; and
(4) turning on the heater and the oxygen supply machine to ferment the tea leaves again until the fermentation of the tea leaves in the tray is completed.

The present disclosure has the beneficial effects that a placement assembly is provided with a support frame and a telescopic connection mechanism, a tray is placed on the support frame, and after the tea leaves are placed in the tray, the tray is placed on the support frame to facilitate the taking and placing of the tea leaves. The whole structure of the support frame is telescopic. The extended support frame is convenient for layered placement of the tea leaves, and the retracted support is convenient for the top cover to make contact with the laminate, the two adjacent laminates to make contact with each other, and the laminate to make contact with the turnover plate. After the support frame is overturned by 180 degrees, positions of the tea leaves at the upper layer and the lower layer are interchanged, at this time, the tea leaves at the lower layer in the tray is overturned to the upper layer to make sufficient contact with the oxygen, thus facilitating the fermentation. The providing of the gas holes in the turnover plate, the laminate and the top cover is convenient for the circulation of the gas in the whole tank body.

### BRIEF DESCRIPTION OF THE DRAWINGS

FIG. 1 is a three-dimensional view of the present disclosure;

FIG. 2 is a front view of the present disclosure;

FIG. 3 is a left view of the present disclosure;

FIG. 4 is a front view of the present disclosure after a tank door is removed;

FIG. 5 is a front view of a placement assembly;

FIG. 6 is a three-dimensional view of a support frame;

FIG. 7 is a top view of a support frame;

FIG. 8 is a top view of a tray;

FIG. 9 is a left view of a support frame;

FIG. 10 is a partial enlarged view of position A in FIG. 9;

FIG. 11 is a left assembly view of a driving member and a slider;

FIG. 12 is a three-dimensional view of a support frame after retracting;

FIG. 13 is a left view of FIG. 12;

FIG. 14 is a top view of a top cover;

FIG. 15 is a diagram of a turnover process of a support frame;

FIG. 16 is a schematic diagram showing position interchange of tea leaves at an upper layer and tea leaves at a lower layer in a tray after a top cover and a laminate of the support frame are overturned;

FIG. 17 is a schematic diagram of an elastic sheet arranged between two connecting rods of a connecting rod group.

In the drawings: 1—tank body; 11—wheel stand; 12—roller; 13—tank door; 14—first box body; 15—second box body; 2—turnover plate; 21—support shaft; 22—gas hole; 23—placement groove; 24—chute; 25—magnet; 3—laminate; 4—top cover; 41—groove; 5—tray; 51—iron sheet; 6—motor; 7—connecting rod; 71—hinge shaft; 72—driving member; 73—slider; 74—elastic sheet; 8—tea leaves at upper layer; 9—tea leaves at lower layer.

### DETAILED DESCRIPTION OF THE EMBODIMENTS

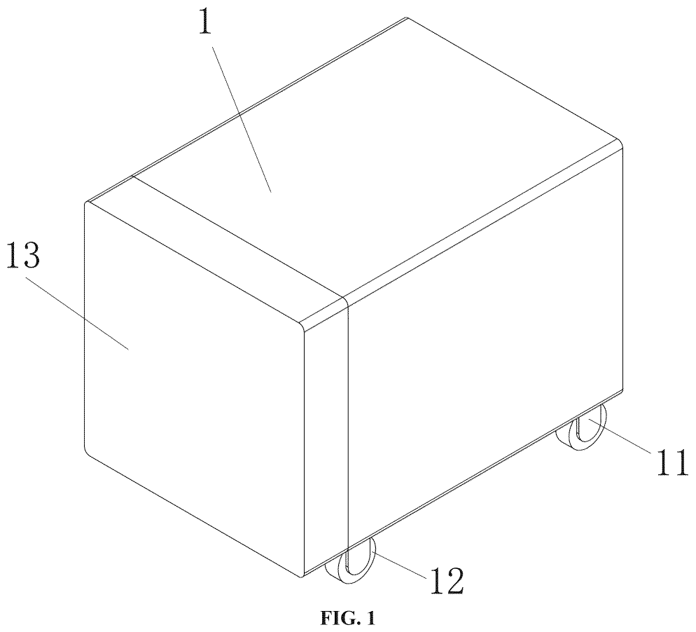

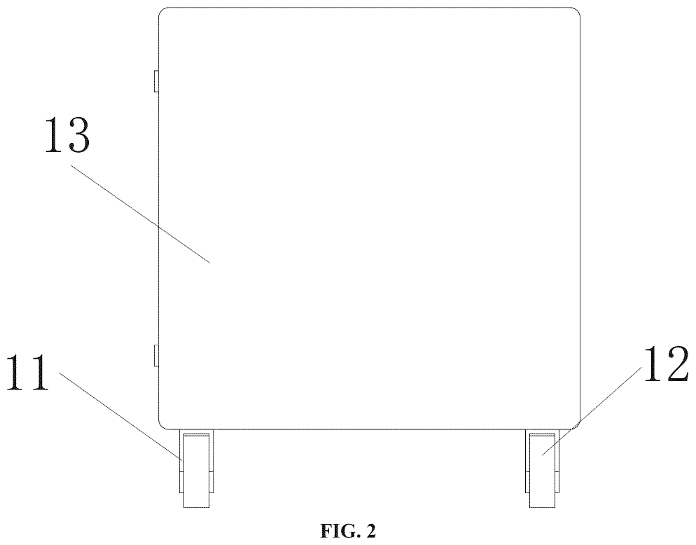

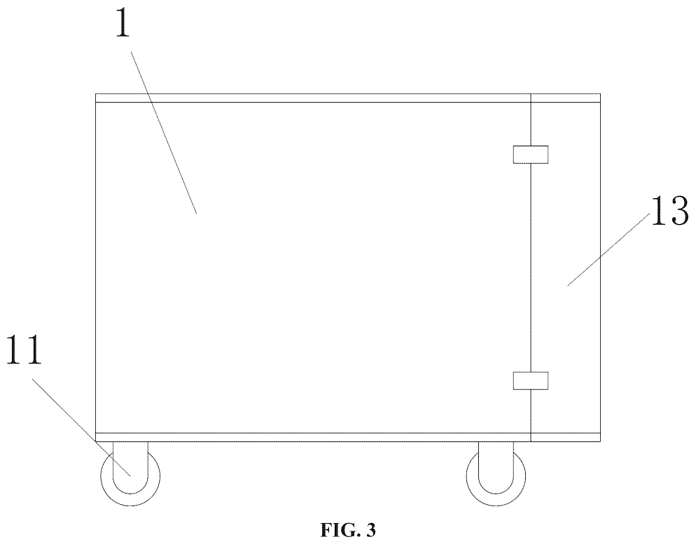

As shown in FIG. 1 to FIG. 17, the present disclosure includes a tank body 1, a placement assembly, a heater, an oxygen supply machine, a temperature sensor, and a humidity sensor. The present disclosure is described in detail below with reference to the accompanying drawings.

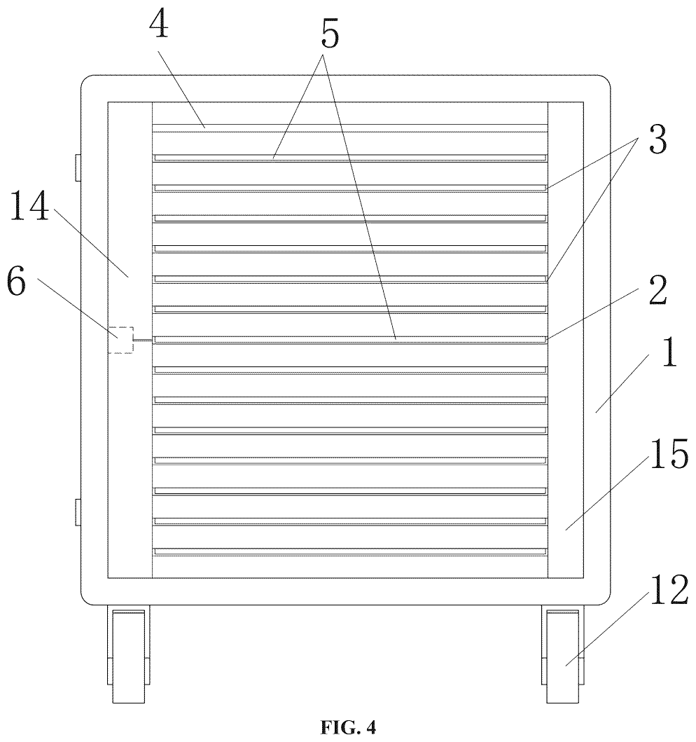

As shown in FIG. 1 to FIG. 17, the multi-layer fermentation tank includes a tank body 1, a placement assembly, a heater, and an oxygen supply machine. A front wall of the tank body 1 is provided with a tank door 13 convenient for open and close. As shown in FIG. 1 to FIG. 3, the bottom of the tank body 1 is provided with a wheel stand 11, and the wheel stand 11 is provided with rollers 12 to facilitate the movement of the whole tank body 1. As shown in FIG. 4, the tank body 1 is internally provided with a first box body 14 and a second box body 15 arranged left and right. The heater and the oxygen supply machine are arranged in the first box body 14 or the second box body 15, for example, the heater can be arranged in the first box body 14, and the oxygen supply machine can be arranged in the second box body 15. The heater is used to heat the inside of the tank body 1, and the oxygen supply machine is used to supply oxygen to the tank body 1. As required, other components for fermenting tea leaves may also be placed in the first box body 14 or the second box body 15, e.g., a humidifier.

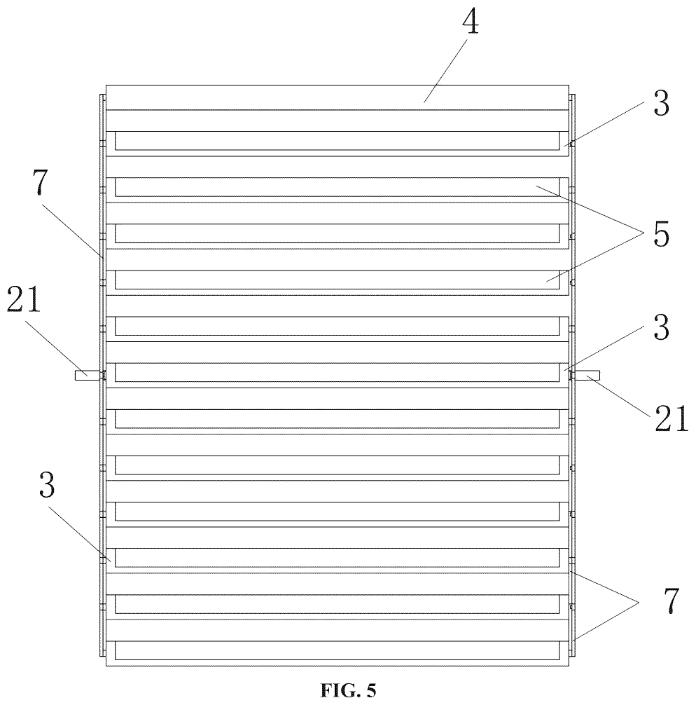

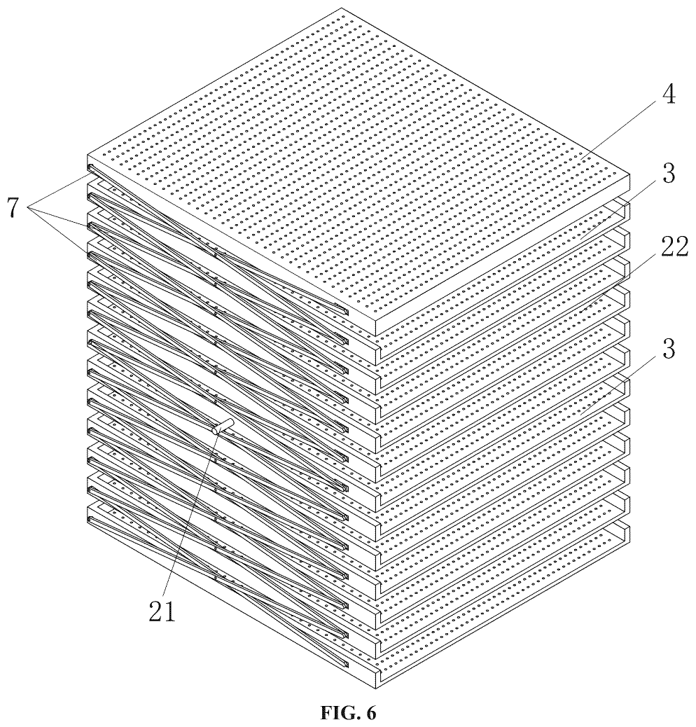

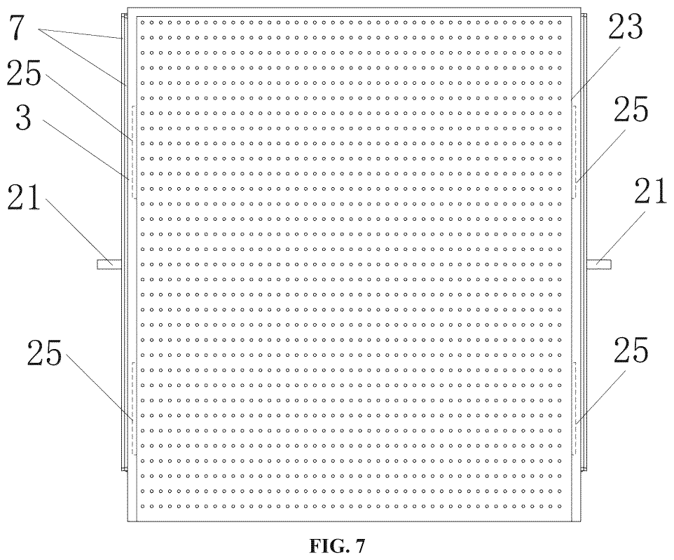

As shown in FIG. 5, the placement assembly includes a support frame, and a tray 5. The support frame is located between the first box body 14, and the second box body 15. As shown in FIG. 6 and FIG. 7, the support frame includes a turnover plate 2, laminate groups arranged on upper and lower sides of the turnover plate 2, and a top cover 4 arranged above the laminate group at the upper side. A telescopic connection mechanism is arranged among the turnover plate 2, the laminate group 2 and the top cover 4. The laminate group includes multiple laminates 3 arranged in sequence. Under the action of the telescopic connection mechanism, a spacing between the turnover plate 2 and the laminate 3 of the laminate group, a spacing between two adjacent laminates 3, and a spacing between the laminate 3 and the top cover 4 are the same. Two telescopic connection mechanisms arranged left and right are provided, and the support frame is located between the two telescopic connection mechanisms.

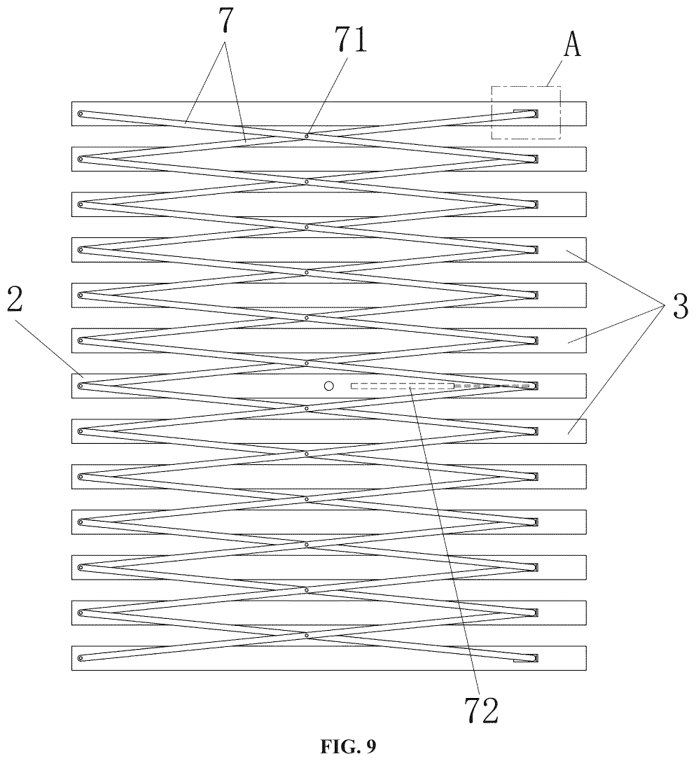

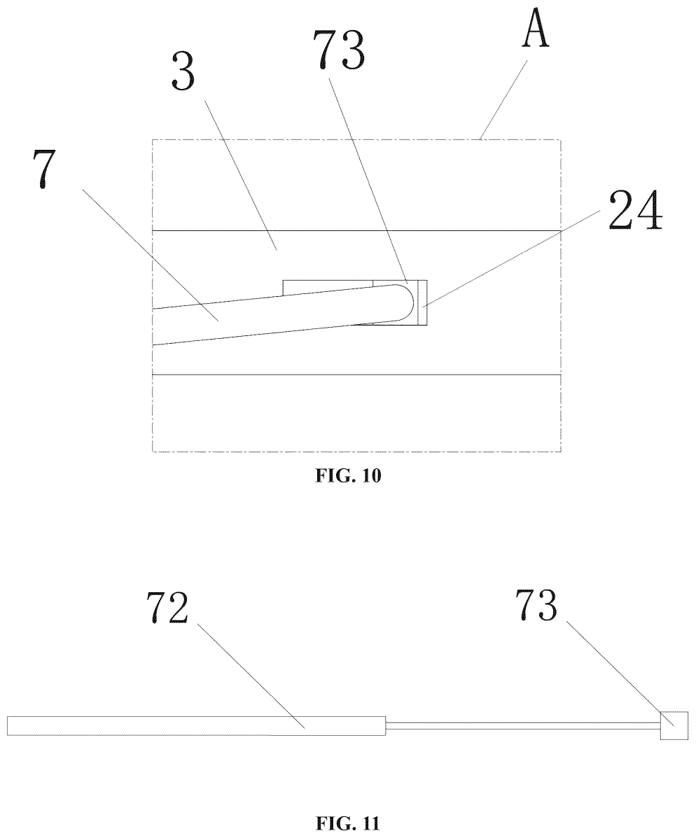

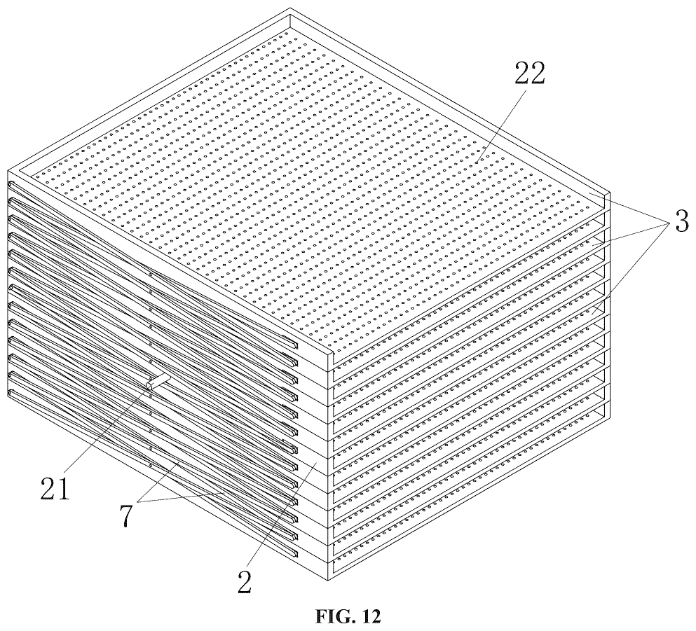

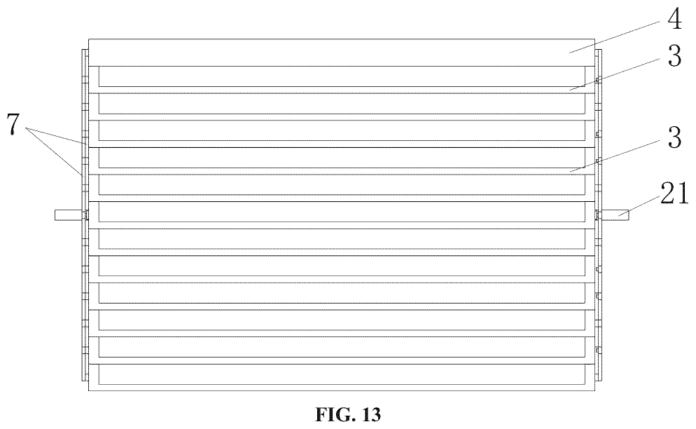

As shown in FIG. 9, the telescopic connection mechanism includes a connecting rod group, and a driving member 72. Multiple connecting rod groups are arranged in sequence. As shown in FIG. 9, each connecting rod group includes two connecting rods 7 which are crossed and hinged by a hinge shaft 71. Corresponding end portions of two adjacent connecting rod groups are hinged, an end portion of one connecting rod 7 of the connecting rod group is hinged with the corresponding top cover 4, laminate 3, or turnover plate 2, and an end portion of the other connecting rod 7 in the connecting rod group is in sliding connection with the corresponding top cover 4, laminate 3, or turnover plate 2. Specifically, the end portion of the other connecting rod 7 of the connecting rod group is hinged with a slider 73 by a hinge shaft 71. As shown in FIG. 9 and FIG. 10, each of left and right side walls of the top cover 4, the laminate 3 or the turnover plate 2 is provided with a chute 24, and the slider 73 is in sliding connection with the corresponding chute 24. The driving member 72 is arranged on the turnover plate 2, and used to drive two connecting rods 7 on the connecting rod group to swing relative to each other and maintain the two connecting rods 7 at a fixed included angle. As shown in FIG. 11, the driving member 72 is an electric telescopic rod, a main body of the driving member 7 is fixed into a side wall of the turnover plate 2, and the slider 73 in sliding connection with the turnover plate 2 is fixed to a telescopic rod of the driving member 72. When the driving member 72 extends and retracts, the slider 73 is driven to move in the chute 24 on the side wall of the turnover plate 2, thus changing the included angle between two connecting rods 7 of the connecting rod group. As shown in FIG. 12 and FIG. 13, when the telescopic rod of the driving member 72 extends to the maximum length, the included angle between the two connecting rods 7 of the connecting rod group is minimum, and at the moment, the top cover 4 is in contact with the laminate 3, the two adjacent laminates 3 are in contact with each other, and the laminate 3 is in contact with the turnover plate 2.

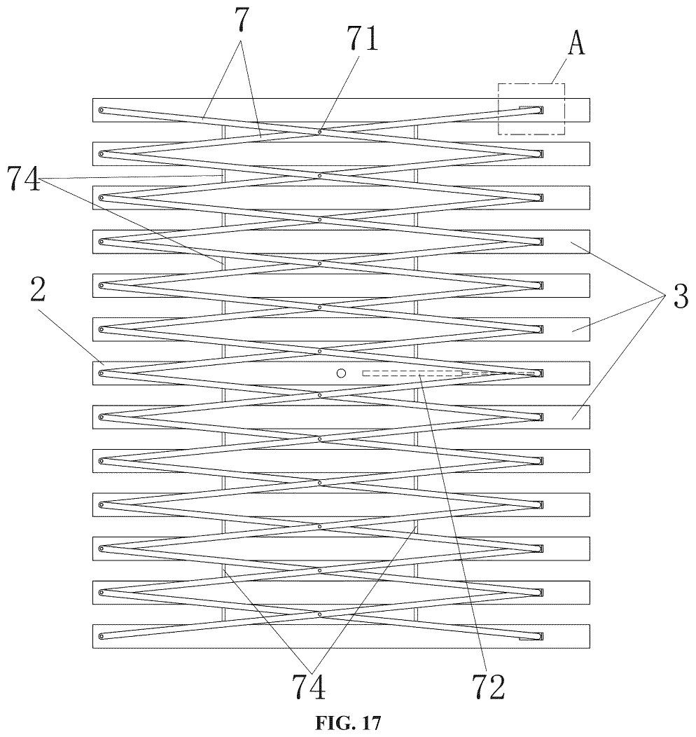

As shown in FIG. 17, an elastic sheet 74 may also be arranged between the two connecting rods 7 of the connecting rod group. Therefore, under the action of the elastic sheet 74, the smooth reset of the two connecting rods 7 of the connecting rod group can be ensured when driving member 72 is reset.

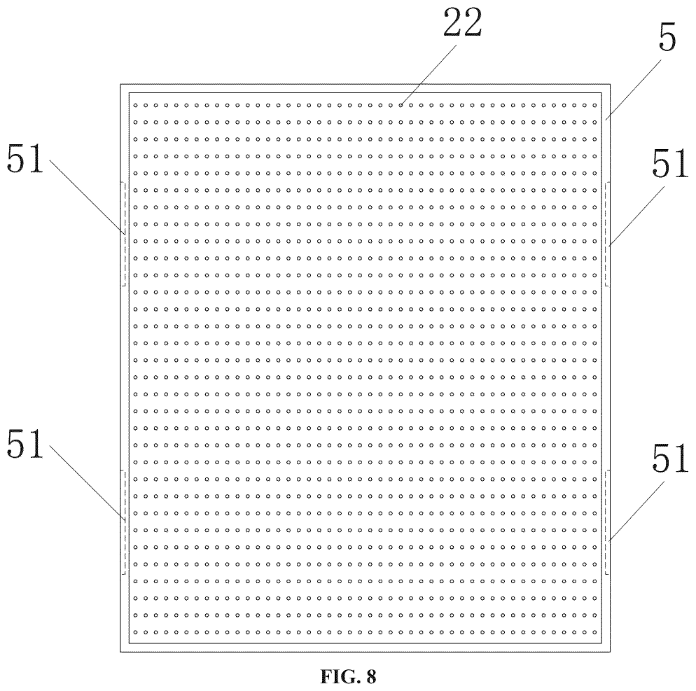

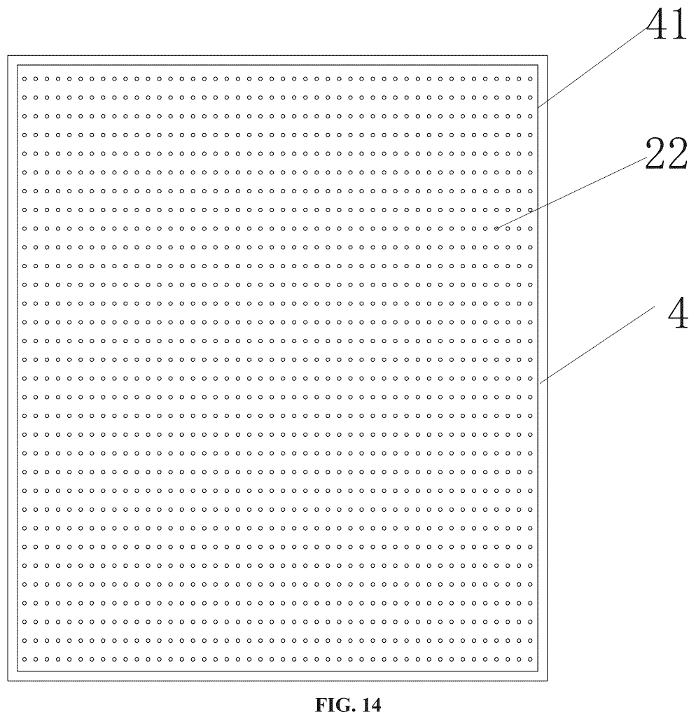

As shown in FIG. 6 and FIG. 7, an upper surface of each of the turnover plate 2 and the laminate 3 is provided with a placement groove 23, the tray 5 is placed in the placement groove 23, and a positioning member is provided between the tray 5 and the placement groove 23. The positioning member plays a role in keeping the tray 5 and the turnover plate 2 relatively fixed, and the tray 5 and the laminate 3 relatively fixed. As shown in FIG. 7 and FIG. 8, the positioning member includes a magnet 25, and an iron sheet 51. The magnet 25 is embedded in an inner wall of the placement groove 23, the iron sheet 51 is embedded in an outer wall of the tray 5, and the iron sheet 51 is in contact and attracted with the corresponding magnet 25 after the tray 5 is placed into the placement groove 23. As shown in FIG. 14, the bottom of the top cover 4 is provided with a square groove 41, and the groove 41 is provided to receive the tea leaves on the laminate 3 adjacent to the top cover 4 after the support frame is overturned by 180 degrees.

As shown in FIG. 6 to FIG. 8 and FIG. 14, gas holes are formed in each of the turnover plate 2, the laminate 3, the tray 5, and the top cover 4. The gas hole 22 is provided to facilitate the oxygen to pass through the turnover plate 2, the laminate 3, the tray 5 and the top cover 4, thus facilitating the oxygen to flow in the tank body 1.

As shown in FIG. 4, the tank body 1 is internally provided with a motor for driving the turnover plate 2 to rotate in a vertical plane. As shown in FIG. 6 and FIG. 7, each of left and right side walls of the turnover plate 2 is provided with a support shaft 21. As shown in FIG. 4, the support shaft 21 at a left end of the turnover plate 2 is rotatably connected to the first box body 14, and the support shaft 21 at a right end of the turnover plate 2 is rotatably connected to the second box body 15. The motor 6 is arranged in the first box body 14, and has an output end fixedly connected to the support shaft 21 at the left end of the turnover plate 2. After the motor 6 is turned on, the motor 6 drives the support shaft 21 at the left end of the turnover plate 2 to rotate, thus driving the rotation of the support frame.

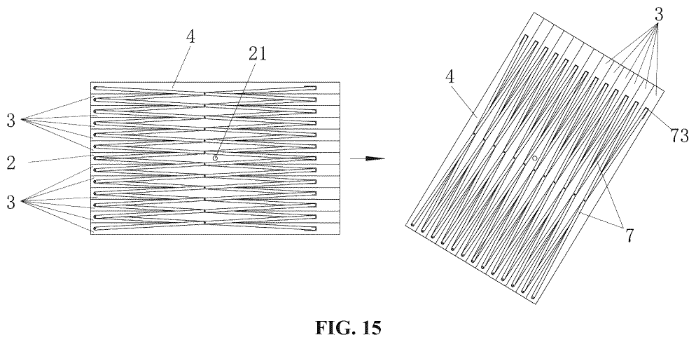

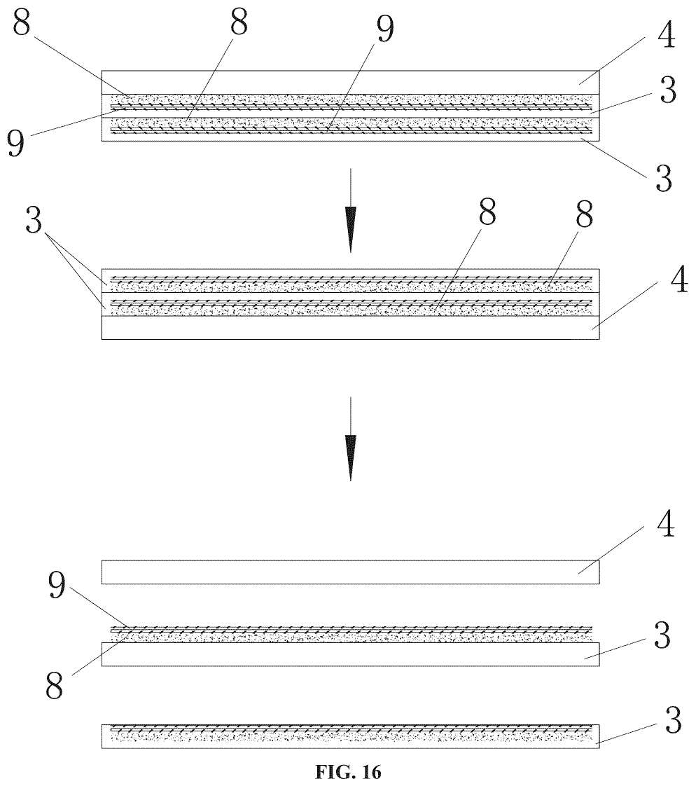

The present disclosure further provides a use method of a multi-layer fermentation tank for tea leaves, including the following steps:
(1) Tea leaves are placed in a tray 5, the tray is placed on each of a laminate 3 and a turnover plate 2, and a tank door 13 is closed.
(2) A heater and an oxygen supply machine are turned on to ferment the tea leaves.
(3) After fermenting for a period of time, a driving member 72 is used to drive a connecting rod group to swing, as shown in FIG. 12, such that a top cover 4 makes contact with the laminate 3, two adjacent laminates 3 are in contact with each other, and the laminate 3 is in contact with the turnover plate 2, as shown in FIG. 15, a motor 6 is turned on to overturn a support frame by 180 degrees. The driving member 72 then drives the top cover 4 to separate from the laminate 3, the two adjacent laminates 3 to separate from each other, and the laminate 3 to separate from the turnover plate 2, as shown in FIG. 15 and FIG. 16, thus interchanging positions of the tea leaves in the tray 5 up and down.
(4) The heater and the oxygen supply machine are turned on to ferment the tea leaves again until the fermentation of tea leaves in the tray is completed.

The present disclosure has the beneficial effects that the placement assembly is provided with the support frame and the telescopic connection mechanism, the tray is placed on the support frame, and after the tea leaves are placed in the tray, the tray is placed on the support frame to facilitate the taking and placing of the tea leaves. The whole structure of the support frame is telescopic. The extended support frame is convenient for layered placement of the tea leaves, and the retracted support is convenient for the top cover to make contact with the laminate, the two adjacent laminates to make contact with each other, and the laminate to make contact with the turnover plate. After the support frame is overturned by 180 degrees, positions of the tea leaves at the upper layer and the lower layer are interchanged, at this time, the tea leaves at the lower layer in the tray is overturned to the upper layer to make sufficient contact with the oxygen, thus facilitating the fermentation. The providing of the gas holes in the turnover plate, the laminate and the top cover is convenient for the circulation of the gas in the whole tank body.

What is claimed is:

1. A multi-layer fermentation tank for tea leaves, comprising:
a tank body having a front wall provided with a tank door,
a placement assembly comprising a support frame and a tray,
a heater arranged in the tank body and being used to heat the inside of the tank body, and
an oxygen supply machine arranged in the tank body and being used to supply oxygen to the tank body,
the support frame comprises a turnover plate, laminate groups located at upper and lower sides of the turnover plate, and a top cover located above the laminate group at the upper side;
a telescopic connection mechanism is arranged among the turnover plate, the laminate and the top cover;
an upper surface of each of the turnover plate and the laminate is provided with a placement groove, the tray is located in the placement groove, and a positioning member is arranged between the tray and the placement groove;
gas holes are formed in each of the turnover plate, the laminate, the tray and the top cover; and
a motor arranged on the tank body drives the turnover plate to rotate in a vertical plane.

2. The multi-layer fermentation tank for tea leaves according to claim 1, wherein the tank body is internally provided with a first box body and a second box body arranged left and right, the support frame is located between the first box body and the second box body, and each of left and right side walls of the turnover plate are provided with a support shaft;
the support shaft at a left end of the turnover plate is rotatably connected to the first box body, the support shaft at a right end of the turnover plate is rotatably connected to the second box body, and the motor is arranged in the first box body, and has an output end fixedly connected to the support shaft at the left end of the turnover plate.

3. The multi-layer fermentation tank for tea leaves according to claim 1, wherein two telescopic connection mechanisms are arranged left and right, and the support frame is located between the two telescopic connection mechanisms.

4. The multi-layer fermentation tank for tea leaves according to claim 3, wherein each of the two telescopic connection mechanism comprises a connecting rod group, and a driving member;
multiple connecting rod groups are arranged in sequence, and each connecting rod group comprises two connecting rods that are crossed and hinged;
corresponding end portions of two adjacent connecting rod groups are hinged, an end portion of one connecting rod of the connecting rod group is hinged with the corresponding top cover, laminate, or turnover plate, and an end portion of the other connecting rod in the connecting rod group is in sliding connection with the corresponding top cover, laminate, or turnover plate; and
the driving member is arranged on the turnover plate to drive the two connecting rods of the connecting rod group to swing relative to each other and maintain the two connecting rods at a fixed included angle.

5. The multi-layer fermentation tank for tea leaves according to claim 4, wherein an end portion of the other connecting rod of the connecting rod group is hinged with a slider, each of left and right side walls of the top cover, the laminate or the turnover plate is provided with a chute, and the slider is in sliding connection with the corresponding chute.

6. The multi-layer fermentation tank for tea leaves according to claim 1, wherein the driving member is an electric telescopic rod, having a main body fixed into a side wall of the turnover plate, and the slider in sliding connection with the turnover plate is fixed to the electric telescopic rod of the driving member.

7. The multi-layer fermentation tank for tea leaves according to claim 1, wherein the positioning member comprises a magnet embedded in an inner wall of the placement groove, and an iron sheet embedded in an outer wall of the tray, and being in contact and attracted with a corresponding magnet after the tray is placed into the placement groove.

8. The multi-layer fermentation tank for tea leaves according to claim 1, wherein the bottom of the top cover is provided with a square groove.

9. The multi-layer fermentation tank for tea leaves according to claim 1, wherein an elastic sheet is provided between two connecting rods of the connecting rod groups.

10. A use method of the multi-layer fermentation tank for tea leaves according to claim 1, the method comprising:
(1) placing tea leaves on the tray, placing the tray on each of the laminate and the turnover plate, and closing the tank door;
(2) turning on the heater and the oxygen supply machine to ferment the tea leaves;
(3) after fermenting for a period of time, driving, by a driving member, a connecting rod group to swing to make a top cover in contact with the laminate, two adjacent laminates in contact with each other, and the laminate in contact with the turnover plate, and turning on a motor to overturn a support frame by 180 degrees; then driving, by the driving member, the top cover to separate from the laminate, the two adjacent laminates to separate from each other, and the laminate to separate from the turnover plate, thus interchanging positions of the tea leaves in each tray up and down; and
(4) turning on the heater and the oxygen supply machine to ferment the tea leaves again until the fermentation of the tea leaves in the tray is completed.

11. The use method according to claim 10, wherein the tank body is internally provided with a first box body and a second box body arranged left and right, the support frame is located between the first box body and the second box body, and each of left and right side walls of the turnover plate is provided with a support shaft; the support shaft at a left end of the turnover plate is rotatably connected to the first box body, the support shaft at a right end of the turnover plate is rotatably connected to the second box body, and the motor is arranged in the first box body, and has an output end fixedly connected to the support shaft at the left end of the turnover plate.

12. The use method according to claim 10, wherein two telescopic connection mechanisms are arranged left and right, and the support frame is located between the two telescopic connection mechanisms.

13. The use method according to claim 12, wherein the telescopic connection mechanism comprises:
a connecting rod group, and
a driving member;
multiple connecting rod groups are arranged in sequence, and each connecting rod group comprises two connecting rods which are crossed and hinged; corresponding end portions of two adjacent connecting rod groups are hinged, an end portion of one connecting rod of the connecting rod group is hinged with the corresponding top cover, laminate, or turnover plate, and an end portion of the other connecting rod in the connecting rod group is in sliding connection with the corresponding top cover, laminate, or turnover plate; and the driving member is arranged on the turnover plate to drive the two connecting rods of the connecting rod group to swing relative to each other and maintain the two connecting rods at a fixed included angle.

14. The use method according to claim 13, wherein an end portion of the other connecting rod of the connecting rod group is hinged with a slider, each of left and right side walls of the top cover, the laminate or the turnover plate is provided with a chute, and the slider is in sliding connection with the corresponding chute.

15. The use method according to claim 10, wherein the driving member is an electric telescopic rod, a main body of the driving member is fixed into a side wall of the turnover plate, and the slider in sliding connection with the turnover plate is fixed to a telescopic rod of the driving member.

16. The use method according to claim 10, wherein the positioning member comprises a magnet, and an iron sheet, the magnet is embedded in an inner wall of the placement groove, the iron sheet is embedded in an outer wall of the tray, and the iron sheet is in contact and attracted with a corresponding magnet after the tray is placed into the placement groove.

17. The use method according to claim 10, wherein the bottom of the top cover is provided with a square groove.

18. The use method according to claim 10, wherein an elastic sheet is provided between two connecting rods of the connecting rod groups.
